(19) United States
(12) Patent Application Publication
(10) Pub. No.: US 2025/0182779 A1
(43) Pub. Date: Jun. 5, 2025

(54) METHOD FOR PROVIDING AUXILIARY INFORMATION ON DYSPHAGIA BY USING VOICE ANALYSIS

(71) Applicants: SEOUL NATIONAL UNIVERSITY HOSPITAL, Seoul (KR); RS REHAB CO., LTD., Seongnam-si, Gyeonggi-do (KR)

(72) Inventors: Ju Seok RYU, Seongnam-si, Gyeonggi-do (KR); Jun Chang LEE, Seongnam-si, Gyeonggi-do (KR)

(21) Appl. No.: 18/727,069

(22) PCT Filed: Jan. 10, 2023
(86) PCT No.: PCT/KR2023/000460

§ 371 Date: Jul. 5, 2024

(30) Foreign Application Priority Data
Jan. 11, 2022 (KR) 10-2022-0004022

(51) Int. Cl. G10L 25/66; A61B 5/00

(52) U.S. Cl. CPC G10L 25/66; A61B 5/4205; A61B 5/4803

(57) ABSTRACT

A method for providing auxiliary information on dysphagia by using voice analysis, the method comprising: obtaining, via a sensor array, the pre-food swallowing voice or vibration and the post-food swallowing voice or vibration of the subject, respectively, and providing auxiliary information on dysphagia by comparing one or more characteristic values of each of said voice or vibration, wherein said auxiliary information on dysphagia comprises characteristic information about the residue state concerning one or more of the following: whether food has been aspirated into the airway after food swallowing by the subject, the presence or absence of residues, the location of residues, and the amount of residues.

### TECHNICAL FIELD

The present application relates to a method for providing auxiliary information on dysphagia using voice analysis.

### CROSS REFERENCE TO RELATED APPLICATIONS

This application claims priority to Korean Patent Application No. 10-2022-0004022, filed on Jan. 11, 2022, the entire contents of which are incorporated herein by reference.

### BACKGROUND

Swallowing is a complex process in which movement from the oral cavity to the esophagus is finely controlled within a short period of time so that food is safely and effectively delivered to the stomach, requiring sequential and well-conditioned contraction and relaxation of the oral and pharyngeal muscles. In this regard, dysphagia (difficulty) refers to the difficulty of swallowing caused by an abnormality in the muscular nervous system or a structural abnormality in the section from the oral cavity to the upper esophagus.

As such, dysphagia can cause various problems, and in particular, because the pharynx through which food passes and the larynx through which air passes coexist anatomically, when swallowing disorder occurs, food is inhaled into the airway through the larynge instead of the esophagus, which increases the risk of inhalation pneumonia secondaryly. Typical methods for determining dysphagia include videofluoro-scopic examination of swallowing (VFS) and Fiberoptic Endoscopic Evaluation of Swallowing (FEES). In particular, with the recent spread of ‘social distancing’ due to the COVID-19 pandemic, ‘untact consumption’ has become commonplace, and ‘untact society’ has emerged as a new paradigm. Accordingly, it is necessary to review the introduction of telemedicine treatment at a comprehensive level. Further, the existing method for diagnosing dysphagia has a problem in that there is a restriction on the economic performance and accessibility of the test equipment through videofluoro-scopic contrast examination of swallowing and endoscopy, that is, a restriction on the movement of the test equipment, and the restriction that the patient should visit the test room causes inconvenience to the patient, and the evaluation is made by the subjective judgment of the evaluator.

### DETAILED DESCRIPTION OF INVENTION

### Technical Problem

The present application is intended to solve the above-described problems, and to provide a method of obtaining, via a sensor array, the pre-food swallowing voice or vibration and the post-food swallowing voice or vibration of the subject, respectively, and providing auxiliary information on dysphagia by comparing one or more characteristic values of each of said voice or vibration.

In another aspect, the present application is intended to provide auxiliary information on the location or amount of the residue, in addition to aspiration.

### Means for Solving the Problem

According to an embodiment of the present application, a method for providing auxiliary information on dysphagia using voice analysis is method for providing auxiliary information on dysphagia using voice analysis, the method comprising: obtaining, via a sensor array, the pre-food swallowing voice or vibration and the post-food swallowing voice or vibration of the subject, respectively, and providing auxiliary information on dysphagia by comparing one or more characteristic values of each of said voice or vibration; wherein said auxiliary information on dysphagia may comprise characteristic information about the residue state concerning one or more of the following: whether food has been aspirated into the airway after food swallowing by the subject, the presence or absence residues, the location of residues, and the amount of residues.

According to an embodiment, each of said sensors of the sensor array is attached to a residue site of interest of the subject, and said step may comprise, calculating one or more characteristic values from each of the pre-food swallowing voice or vibration and the post-food swallowing voice or vibration of the subject obtained by each of the sensors by sites on the subject; and comparing one or more characteristic values of each of said pre-swallowing voice or vibration and post-swallowing voice or vibration obtained at the same site, to calculate characteristic information on the residue state, comprising one or more of following: whether food has been aspirated into the airway after the subject's food swallowing, the presence or absence of residues, the site of residues, and the amount of residues.

According to an embodiment, each of the sensors of said sensor array may comprise at least a microphone.

According to an embodiment, each of the sensors of said sensor array may further comprise a vibration sensor that senses vibration.

According to an embodiment, said method may further provide information on one or more of the following: whether food has been aspirated into the airway and whether residues remain in the pharynx, based on the characteristic information about said residue state.

According to an embodiment, said method may further comprise: determining, based on said characteristic information on the residue state, whether food has been aspirated into the airway of the subject after the subject's food swallowing; and determining, in response to determining that food has been aspirated into the airway of the subject, the amount of the aspirated food based on said characteristic information on the residue state.

According to an embodiment, said step of determining whether food has been aspirated into the airway of the subject may determine that food has been aspirated into the airway if the difference between one or more characteristic values of each of the pre-food swallowing voice or vibration and the post-food swallowing voice or vibration of the subject obtained by a sensor attached to the subject's oral cavity or airway proximal site is greater than or equal to a pre-specified first reference value.

According to an embodiment, said method may further comprise: determining, based on said characteristic information on the residue state, whether residues remain in the pharynx of the subject after the subject's food swallowing; and determining, in response to determining that residues remain in the pharynx of the subject, the amount of residues remaining in the pharynx based on said characteristic information on the residue state.

According to an embodiment, said step of determining whether residues remain in the pharynx of the subject may determine that residues remain in the pharynx of the subject if the difference between one or more characteristic values of the pre-food swallowing voice or vibration and the post-food swallowing voice or vibration of the subject obtained by the sensor attached to the subject's oral cavity or pharynx proximal site is greater than or equal to a pre-specified second reference value.

According to an embodiment, said voice or vibration may be a voice or vibration when the subject utters a specific word for a period of time.

According to an embodiment, said one or more characteristic values may be calculated using a PRAAT.

According to an embodiment, said one or more characteristic values may comprise one or more selected from the group consisting of average fundamental frequency (F0) for all extracted pitch periods of voice or vibration, standard deviation of said fundamental frequency, relative average perturbation (RAP), jitter, shimmer percentage, amplitude perturbation quotient (APQ), noise-to-harmonic ratio (NHR), harmonics to noise ratio (HNR), voice turbulence index (VTI), and signal to noise ratio (SNR).

According to an embodiment, said relative average perturbation may be the variability of the pitch period in the voice or vibration sample analyzed at a smoothing factor of 3 cycles.

According to an embodiment, said voice turbulence index may be the relative energy level of high frequency noise.

According to an embodiment, if the relative average perturbation (RAP) of the post-food swallowing voice or vibration increases or decreases compared to said pre-food swallowing voice or vibration obtained by the sensor attached to the same site, the residue location may be determined to be near that sensor attachment site, and it may be determined that the amount of residue is large in the region close to the site where said RAP variability is greatest among the sites to which the sensor is attached, by comparing the RAP variability of said pre-food swallowing voice or vibration and post-food swallowing voice or vibration obtained by each of the sensors by sites on the subject.

According to an embodiment, if at least one of jitter, shimmer percentage, and voice turbulence index of the post-swallowing voice or vibration increases or decreases compared to said pre-food swallowing voice or vibration, the residue location may be determined to be near that sensor attachment site, and it may be determined that that the amount of residue is large in the region close to the site where said variability is greatest among the sites to which the sensor is attached, by comparing the variability of at least one of jitter, shimmer percentage, and voice turbulence index of said pre-food swallowing voice or vibration and post-food swallowing voice or vibration obtained by each of the sensors by sites on the subject.

According to an embodiment, said residue sites of interest may be at least one or more of the subject's lips, the skin surface near the vallecular pouch, the skin surface near the vocal cord, the skin surface near the pyriformis sinus, and the skin surface near the pharyngeal wall, of the subject's neck.

According to an embodiment, said method may further comprise: providing control information for an electrical stimulator to assist with dysphagia rehabilitation treatment of the subject, or providing guide information about food if the characteristic information on the residue state is determined to be within a pre-set range related to dysphagia, by obtaining the pre-food swallowing voice or vibration and the post-food swallowing voice or vibration of the subject via the sensor array, respectively, and comparing the one or more characteristic values of each of said voice or vibration.

According to an embodiment, said method may provide control information for the parameter values of a 4-channel electrostimulator, based on at least one of whether food has been aspirated into the airway, the amount of the aspirated food, whether residues remain in the pharynx, and the amount of residue remaining in the pharynx.

According to an embodiment, the method may provide guide information on the type, quality, and concentration of food, based on at least one of whether food has been aspirated into the airway, the amount of the aspirated food, whether residues remain in the pharynx, and the amount of residue remaining in the pharynx.

According to an embodiment, the subject is a dysphagia patient, and the auxiliary information on dysphagia comprises auxiliary information on the improvement of dysphagia.

According to an embodiment, said method may further comprise obtaining, via the sensor array, at least a first voice or vibration and a second voice or vibration, respectively, after subject's food swallowing, with said second voice or vibration obtained after a period of time has elapsed from said first voice or vibration; and comparing one or more characteristic values of each of the pre-food swallowing voice or vibration, the first post-swallowing voice or vibration, and the second post-swallowing voice or vibration of the subject obtained at the same site to provide characteristic information on the residue state.

According to an embodiment, when the program instructions are executed by a processor of a computer, said processor may be a computer-readable recording medium storing program instructions that are readable by the computer and operable by said computer to cause the processor to perform the method for providing auxiliary information on dysphagia using voice analysis.

### Effect of Invention

According to the method for providing auxiliary information on dysphagia using such voice analysis, it is possible to accurately and effectively calculate auxiliary information, particularly information on the location or amount of residue, for determining whether there is a dysphagia by comparing each characteristic value of voice or vibration before and after the subject's food swallowing.

The effects of the present application are not limited to the effects mentioned above, and other effects not mentioned will be clearly understood by those skilled in the art from the description of the claims.

### BRIEF DESCRIPTION OF THE DRAWINGS

In order to more clearly describe the technical solutions of the embodiments of the present application or the prior art, the drawings required in the description of the embodiments are briefly introduced below. It should be understood that the following drawings are for the purpose of describing embodiments of the present specification only and are not intended to be limiting. In addition, for clarity of description, some elements to which various modifications have been applied, such as exaggeration, omission, etc., may be shown in the drawings below.

FIG. 1 is a diagram illustrating a residue site of interest that causes dysphagia, according to an embodiment of the present application.

FIG. 2 is a diagram illustrating a sensor array that acquires the pre-food swallowing voice or vibration and the post-food swallowing voice or vibration of the subject, respectively, according to an embodiment of the present application.

FIG. 3 is a flowchart of a method for providing auxiliary information on dysphagia using voice analysis, according to an embodiment of the present application.

FIG. 4 is a detailed flowchart of a step of providing information on whether food has been aspirated into the airway, according to an embodiment of this application.

FIG. 5 is a detailed flowchart of a step of providing information on whether residues remain in the pharynx, according to an embodiment of this application.

### EMBODIMENTS FOR IMPLEMENTATION OF THE INVENTION

The terminology used herein is for the purpose of referring only to specific embodiments and is not intended to limit the present application. The singular forms used herein include the plural forms as well, unless the phrases expressly indicate the opposite meaning. The meaning of “comprising” as used herein embodies a particular characteristic, region, integer, step, operation, item, and/or component, and does not exclude the presence or addition of other characteristics, regions, integers, steps, operations, items, and/or components.

Although not defined otherwise, all terms including technical and scientific terms used herein have the same meaning as commonly understood by one of ordinary skill in the art to which the present application belongs. Usually used predefined terms are further interpreted as having a meaning consistent with the relevant technical literature and the presently disclosed content and are not interpreted in an ideal or very formal sense unless defined.

Aspiration in the present specification means that an object to be swallowed, such as food, flows into the organ system in the process of swallowing by an individual.

In the present specification, the residue means that the object to be swallowed, such as food, does not pass to the esophagus in the course of swallowing by the individual, and remains in a site including at least one of Vallecular pouch, vocal cord, pyriformis sinus, and pharyngeal wall.

In the present specification, the auxiliary information on dysphagia may comprise characteristic information about the residue state concerning one or more of the following: whether an object to be swallowed, such as food, has been aspirated into the airway after swallowing the food, etc. of the subject, the presence or absence of a residue, the location of the residue, and the amount of the residue.

In the present specification, the area proximate to a site to which a sensor or sensor array is attached may mean an area within a pre-set location from the site to which the sensor or sensor array are attached.

In the present specification, the sensor array may mean a sensor assembly comprising one or more sensors.

Hereinafter, embodiments of the present application will be described in detail with reference to the drawings.

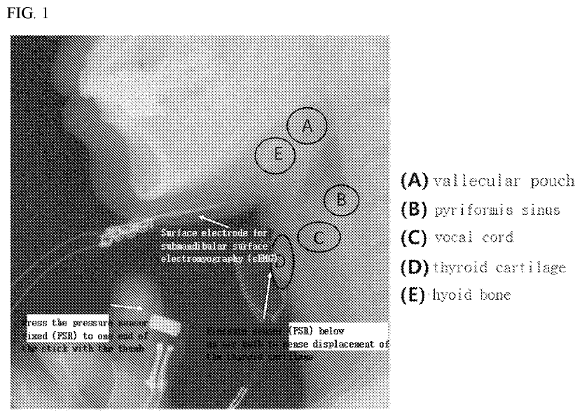

FIG. 1 is a diagram illustrating a residue site of interest that causes dysphagia, according to an embodiment of the present application.

Referring to FIG. 1, a method for providing auxiliary information on dysphagia using voice analysis (hereinafter, “method of providing auxiliary information about dysphagia”) may obtain the pre-food swallowing voice or vibration and the post-food swallowing voice or vibration of the subject, respectively, via a sensor array. Each of the sensors of said sensor array may be attached to a residue site of interest of the subject, wherein said residue site of interest may comprise the subject's lips, the skin surface adjacent to at least one of (A) Vallecular pouch, (B) pyriformis sinus, (C) vocal cord, (D) thyroid cartilage, (E) hyoid bone and a pharyngeal wall of the subject's neck. Additionally, it may further comprise a pressure sensor fixed (FSR) to one end of the stick that can be pressed with the thumb, a surface electrode for submandibular surface electromyography (sEMG), and a pressure sensor (FSR) below an air-bulb to sense displacement of the thyroid cartilage.

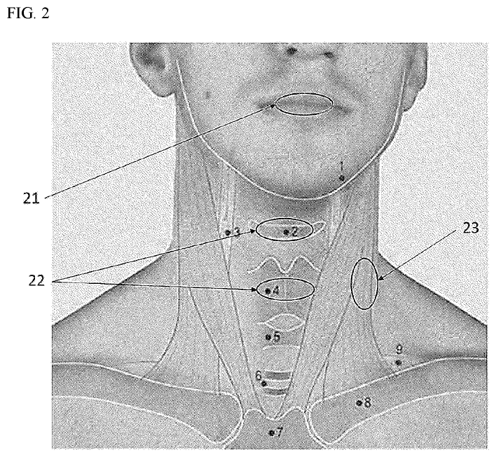

FIG. 2 is a diagram illustrating a sensor array that acquires the pre-food swallowing voice or vibration and the post-food swallowing voice or vibration of the subject, respectively, according to an embodiment of the present application.

Referring to FIG. 2, each of the sensors of said sensor array may be attached to a residue site of interest of the subject and comprise at least a microphone. In an embodiment, said sensor may further comprise a vibration sensor. The first sensor may be attached to the site 21, which may be the lips, or the mouth of the subject. The second sensor or the third sensor may be attached to the site 22 to obtain the voice or vibration before and after food swallowing, and the site 22 is close to the pyriformis sinus or vocal cord.

The method of providing auxiliary information about dysphagia may compare the voice or vibration before and after swallowing obtained by the second sensor or the third sensor attached to the site 22 to calculate the variability of the characteristic information before and after swallowing more strongly when residuals are detected on the vocal cord, thereby providing the auxiliary information about the presence of dysphagia.

The fourth sensor may be attached to the site 23 to obtain voice or vibration before and after swallowing food, and the method of providing auxiliary information about dysphagia may compare the voice or vibration before and after swallowing obtained by the fourth sensor attached to the site 23 to calculate the variability of the characteristic information before and after swallowing more strongly when residuals are detected on the pyriformis sinus, thereby providing the auxiliary information about the presence of dysphagia.

The method of providing auxiliary information about dysphagia may comprise calculating one or more characteristic values from each of the pre-food swallowing voice or vibration and the post-food swallowing voice or vibration of the subject obtained by each of the sensors by sites 21, 22, 23 on the subject, and comparing one or more characteristic values of each of said pre-swallowing voice or vibration and post-swallowing voice or vibration obtained at the same site, to calculate characteristic information on the residue state, comprising one or more of following: whether food has been aspirated into the airway after the subject's food swallowing, the presence or absence of residues, the site of residues, and the amount of residues.

For example, if the relative average perturbation (RAP) of the post-food swallowing voice or vibration increases or decreases compared to said pre-food swallowing voice or vibration obtained by the sensor attached to the same site 22, the residue location may be determined to be in the vocal cord, which is an area proximate to the site 22 to which that sensor is attached. By comparing the RAP variability of said pre-food swallowing voice or vibration and post-food swallowing voice or vibration obtained by each of the sensors by sites 21, 22, 23 on the subject, it can be determined that the amount of residue is largest in the area close to the second sensor when the variability of the relative average perturbation (RAP) of the post-swallowing voice or vibration is the largest in the sensor attached to the site 22 among the sites 21, 22, 23 to which the sensor is attached.

In an embodiment, said voice or vibration obtained by the sensor array may be a voice or vibration when the subject utters a specific word for a period of time. For example, the subject may utter a- or i- for 5 seconds.

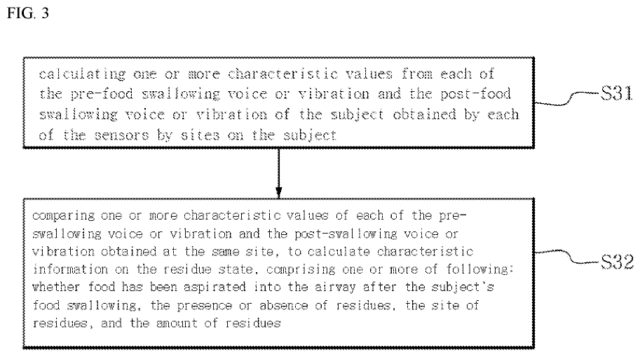

FIG. 3 is a flowchart of a method for providing auxiliary information on dysphagia using voice analysis, according to an embodiment of the present application.

The method of providing auxiliary information about dysphagia may comprise: providing auxiliary information about dysphagia by obtaining the pre-food swallowing voice or vibration and the post-food swallowing voice or vibration of the subject via the sensor array, respectively, and comparing the one or more characteristic values of each of said voice or vibration. Said auxiliary information about dysphagia may comprise characteristic information on the residue state relating to one or more of following: whether food has been aspirated into the airway after the subject's food swallowing, the presence or absence of residues, the site of residues, and the amount of residues.

Referring to FIG. 3, said step may include a step S31 of calculating one or more characteristic values from each of the pre-food swallowing voice or vibration and the post-food swallowing voice or vibration of the subject obtained by each of the sensors by sites on the subject, and a step S32 of comparing one or more characteristic values of each of said pre-swallowing voice or vibration and post-swallowing voice or vibration obtained at the same site, to calculate characteristic information on the residue state, comprising one or more of following: whether food has been aspirated into the airway after the subject's food swallowing, the presence or absence of residues, the site of residues, and the amount of residues.

In an embodiment, said one or more characteristic values may be calculated using a PRAAT. The PRAAT is a voice analysis program that can extract various features from voice signals.

In an embodiment, said one or more characteristic values comprise one or more selected from the group consisting of average fundamental frequency (F0) for all extracted pitch periods of voice or vibration, standard deviation of said fundamental frequency, relative average perturbation (RAP), jitter, shimmer percentage, amplitude perturbation quotient (APQ), noise-to-harmonic ratio (NHR), harmonics to noise ratio (HNR), voice turbulence index (VTI), and signal to noise ratio (SNR).

Said relative average perturbation is the variability of the pitch period in the voice or vibration sample analyzed at the smoothing factor of 3 cycles, and said voice turbulence index is the relative energy level of high frequency noise.

In an embodiment, if the relative average perturbation (RAP) of the post-food swallowing voice or vibration increases or decreases compared to said pre-food swallowing voice or vibration obtained by the sensor attached to the same site, the residue location may be determined to be near that sensor attachment site, and it may be determined that the amount of residue is large in the region close to the site where said RAP variability is greatest among the sites to which the sensor is attached, by comparing the RAP variability of said pre-food swallowing voice or vibration and post-food swallowing voice or vibration obtained by each of the sensors by sites on the subject.

In an embodiment, if at least one of jitter, shimmer percentage, and voice turbulence index of the post-swallowing voice or vibration increases or decreases compared to said pre-food swallowing voice or vibration, the residue location may be determined to be near that sensor attachment site, and it may be determined that that the amount of residue is large in the region close to the site where said variability is greatest among the sites to which the sensor is attached, by comparing the variability of at least one of jitter, shimmer percentage, and voice turbulence index of said pre-food swallowing voice or vibration and post-food swallowing voice or vibration obtained by each of the sensors by sites on the subject.

In one another embodiment, said method of providing auxiliary information about dysphagia may further comprise: obtaining, via a sensor array, the pre-food swallowing voice or vibration and the post-food swallowing voice or vibration of the subject, respectively; and each of the sensors of said sensor array is attached to a residue site of interest of the subject, and calculating characteristic values from the pre-food swallowing voice or vibration and the post-food swallowing voice or vibration of the subject obtained by each of the sensors by sites on the subject.

In an embodiment, at least a first voice or vibration and a second voice or vibration, respectively, may be obtained via the sensor array after subject's food swallowing, wherein said second voice or vibration may be a voice or vibration after a period of time has elapsed from said first voice or vibration. For example, the time at which the second voice or vibration is acquired may be a time that several weeks or months have elapsed since the first voice or vibration is obtained.

Characteristic information on the residue state can be provided by comparing one or more characteristic values of each of the pre-food swallowing voice or vibration, the first post-swallowing voice or vibration, and the second post-swallowing voice or vibration of the subject obtained at the same site. The subject is a dysphagia patient, and this provides auxiliary information on the improvement of dysphagia at the time when the second voice or vibration is obtained by performing dysphagia treatment from the time when the first voice or vibration is obtained. For example, if the magnitude of the variability of the relative average perturbation (RAP) of the voice or vibration before and after swallowing decreases, it may be determined that the dysphagia of the subject is improving.

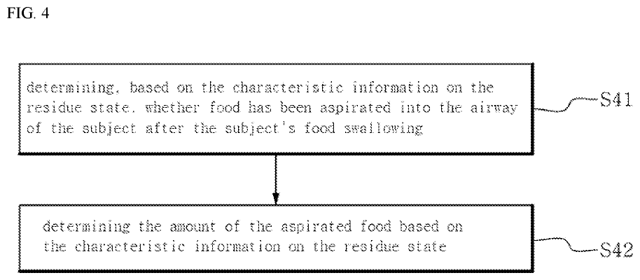

FIG. 4 is a detailed flowchart of a step of providing information on whether food has been aspirated into the airway, according to an embodiment of this application.

Referring to FIG. 4, the step of providing information on whether food has been aspirated into the airway may comprise: a step S41 of determining, based on said characteristic information on the residue state, whether food has been aspirated into the airway of the subject after the subject's food swallowing, and a step S42 of determining, in response to determining that food has been aspirated into the airway of the subject, the amount of the aspirated food based on said characteristic information on the residue state.

In an embodiment, said step of determining whether food has been aspirated into the airway of the subject may determine that food has been aspirated into the airway if the difference between one or more characteristic values of each of the pre-food swallowing voice or vibration and the post-food swallowing voice or vibration of the subject obtained by a sensor attached to the subject's oral cavity or airway proximal site is greater than or equal to a pre-specified first reference value.

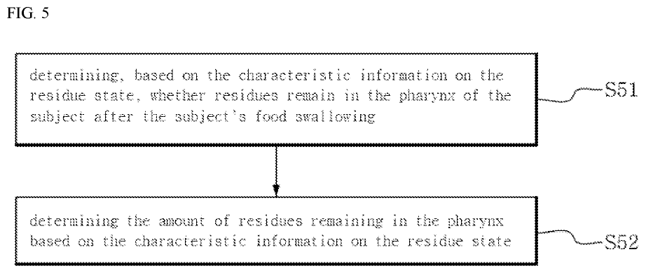

FIG. 5 is a detailed flowchart of a step of providing information on whether residues remain in the pharynx, according to an embodiment of this application.

Referring to FIG. 5, the step of further providing information on one or more of whether residues remain in the pharynx may comprise: a step S51 of determining, based on said characteristic information on the residue state, whether residues remain in the pharynx of the subject after the subject's food swallowing, and a step S52 of determining, in response to determining that residues remain in the pharynx of the subject, the amount of the residue remaining in the pharynx based on said characteristic information on the residue state.

In an embodiment, said step of determining whether residues remain in the pharynx of the subject may determine that residues remain in the pharynx of the subject if the difference between one or more characteristic values of the pre-food swallowing voice or vibration and the post-food swallowing voice or vibration of the subject obtained by the sensor attached to the subject's oral cavity or pharynx proximal site is greater than or equal to a pre-specified second reference value.

The harmonics to noise ratio (HNR) may be used as a characteristic value that is an indicator in the step of determining whether residues remain in the pharynx. The harmonic-to-noise ratio (HNR) of the voice or vibration before and after the subject's food swallowing may be reduced due to noise caused by the residues.

In an embodiment, voice analysis may be used to identify changes in sound or vibration parameters before and after food swallowing.

For example, a videofluoroscopic swallowing study (VFS) may be used to compare the characteristic values of each variable of voice or vibration before and after swallowing of the subjects with dysphagia. Said one or more characteristic values may comprise one or more selected from the group consisting of average fundamental frequency (F0) for all extracted pitch periods of voice or vibration, standard deviation of said fundamental frequency, relative average perturbation (RAP), jitter, shimmer percentage, amplitude perturbation quotient (APQ), noise-to-harmonic ratio (NHR), harmonics to noise ratio (HNR), voice turbulence index (VTI), and signal to noise ratio (SNR).

In an embodiment, said step of determining whether food has been aspirated into the airway of the subject may determine that food has been aspirated into the airway if the difference between one or more characteristic values of each of the pre-food swallowing voice or vibration and the post-food swallowing voice or vibration of the subject obtained is greater than or equal to a pre-specified first reference value.

For example, if the difference between the average fundamental frequency (F0) of voice or vibration before and after food swallowing is in the range of 2.953±24.081, the subject may be classified into a low-risk group, and if the difference is in the range of 9.969±42.965, which is more variable, the subject may also be classified into a high-risk group.

If the difference between the relative average perturbation (RAP) of voice or vibration before and after food swallowing is in the range of −0.0246±02135, the subject may be classified into a low-risk group, and if the difference is in the range of 2.2070±0.2135, which is more variable, the subject may be classified into a high-risk group.

Depending on whether there are the residues or whether there is aspiration, there may be a difference between the ratio of the area of the high-pitched region to the low-pitched region, or the ratio of the area of the high-pitch region to the entire region, etc., as the variability in the relative average perturbation (RAP), the shimmer percentage of the voice (SHIM), and the amplitude of the voice waveform increases.

If the difference between the shimmer percentage of the voice or vibration before and after food swallowing (SHIM) is in the range of 0.3246±2.3772, the subject may be classified into a low-risk group, and if the difference is in the range of 4.4811±4.9568, which is more variable, the subject may be classified into a high-risk group.

If the difference between the noise-to-harmonic ratio (NHR) of voice or vibration before and after food swallowing is in the range of 0.3517±5.642, the subject may be classified into a low-risk group, and if the difference is in the range of 19.48±19.49, which is more variable, the subject may be classified into a high-risk group.

If the difference between the voice turbulence index (VTI) before and after food swallowing is in the range of 0.8092±2.730, the subject may be classified into a low-risk group, and if the difference is in the range of 4.965±8.636, which is more variable, the subject may be classified into a high-risk group.

In an embodiment, in the method of providing auxiliary information about dysphagia, if the relative average perturbation (RAP) of the post-food swallowing voice or vibration increases or decreases compared to said pre-food swallowing voice or vibration obtained by the sensor attached to the same site, the residue location is determined to be near the sensor attachment site, and it may be determined that the amount of residue is large in the region close to the site where said variability of relative average perturbation (RAP) is greatest among the sites to which the sensor is attached, by comparing the variability of relative average perturbation (RAP) of said pre-food swallowing voice or vibration and post-food swallowing voice or vibration obtained by each of the sensors by sites on the subject.

In an embodiment, in the method of providing auxiliary information about dysphagia, if at least one of jitter, shimmer percentage, and voice turbulence index of the post-swallowing voice or vibration increases or decreases compared to said pre-food swallowing voice or vibration, the residue location is determined to be near the sensor attachment site, and it may be determined that the amount of residue is large in the region close to the site where said variability is greatest among the sites to which the sensor is attached, by comparing the variability of at least one of jitter, shimmer percentage, and voice turbulence index of said pre-food swallowing voice or vibration and post-food swallowing voice or vibration obtained by each of the sensors by sites on the subject.

In one additional embodiment, based on the analysis, integrated analysis, and multidimensional analysis between each parameter of the average fundamental frequency (F0) of each of voice or vibration before and after food swallowing, the standard deviation of said fundamental frequency, the relative average perturbation (RAP), the jitter, the shimmer percentage, the amplitude perturbation quotient (APQ), the noise-to-harmonic ratio (NHR), the harmonic to noise ratio (HNR), the voice turbulence index (VTI), and the signal to noise ratio (SNR), the pattern may be extracted for whether food has been aspirated into the airway, the amount of the aspirated food, whether residues remain in the pharynx, and the amount of residue remaining in the pharynx.

In another embodiment of the present application, the method of providing auxiliary information about dysphagia may further comprise: providing control information for an electrical stimulator to assist with dysphagia rehabilitation treatment of the subject, or providing guide information about food if the characteristic information on the residue state is determined to be within a pre-set range related to dysphagia, by obtaining the pre-food swallowing voice or vibration and the post-food swallowing voice or vibration of the subject via the sensor array, respectively, and comparing the one or more characteristic values of each of said voice or vibration.

In an embodiment, the method of providing auxiliary information about dysphagia may provide control information for the parameter values of a 4-channel electrostimulator, based on at least one of whether swallowing food has been aspirated into the airway, the amount of the aspirated food, whether residues remain in the pharynx, and the amount of residue remaining in the pharynx. The 4-channel electrostimulation therapy device assists the subject in swallowing so that when a dysphagia patient consumes food, electrostimulation is applied to a specific site so that no residues remain in the residue sites of interest. The electrostimulation therapy device is one of the facilitation techniques, which may be likened to neuromuscular electrical stimulation or functional electrical stimulation applied to the upper and lower extremities by applying electrical stimulation using a surface electrode to the muscles under the jaw and the occipitalis muscle of the neck region. The mechanisms include changes in the plasticity of the brain due to the provision of somatosensory sensation or the repetitive movement of the occipitalis muscle. In the method of providing auxiliary information about dysphagia, the 4-channel electrostimulation therapy device is located in the airway site to help the subject's swallowing action if it is determined that the swallowing food has been aspirated into the airway. The intensity of the electrostimulation can be adjusted according to the amount of the aspirated food.

In an embodiment, the method of providing auxiliary information about dysphagia may provide guide information on the type, quality, and concentration of food, based on at least one of whether food has been aspirated into the airway, the amount of the aspirated food, whether residues remain in the pharynx, and the amount of residue remaining in the pharynx.

For example, if it is determined that oral intake is possible, the viscosity and texture of the diet are adjusted to suit the patient. In the case of a liquid phase, a starch-based food thickener is mixed to increase viscosity. In general, the lower the viscosity (thinner consistency), the more difficult it is to control the food in the oral cavity and pharynx, so aspiration easily occurs. Conversely, the lower the viscosity, the better it flows through the upper esophageal sphincter, so there is also a point that the bolus formation proceeds easily. Therefore, considering these two aspects, it is necessary to adjust the viscosity according to the results of information on dysphagia via voice analysis according to each patient. As the patient's swallowing function gradually improves, food should be consumed in the order of semi-solid food and normal diet, and the like. It may also provide patients with guide information on the amount that can be swallowed at a time and how many times to swallow.

According to such a method for providing auxiliary information on dysphagia using voice analysis, the present application has an effect of increasing the reliability of evaluation while ensuring safety and simplicity by analyzing the voice or vibration of the subject before and after swallowing food and using it as auxiliary information for determining whether there is dysphagia.

The operation by the method for providing auxiliary information on dysphagia using voice analysis according to the embodiments described above may be at least partially implemented as a computer program and recorded in a computer-readable recording medium. For example, it is implemented with a program product consisting of a computer-readable medium containing program code, which may be executed by a processor to perform any or all steps, operations, or processes described.

The method for providing auxiliary information on dysphagia using voice analysis according to one another aspect of present application may be performed by a computing device including a processor. Said computing device may be a computing device, such as a desktop computer, laptop computer, notebook, smart phone, or the like, or may also be any device capable of being integrated. A computer is an device having one or more alternative and special purpose processors, memory, storage, and networking components (either wireless or wired). Said computer may execute an operating system, such as, for example, an operating system compatible with Microsoft's Windows, Apple's OS X or iOS, Linux distribution, or Google's Android OS.

Said computer-readable recording medium comprises all types of recording identification devices in which data readable by a computer is stored. Examples of computer-readable recording media comprise ROM, RAM, CD-ROM, magnetic tape, floppy disk, optical data storage identification device, and the like. The computer-readable recording medium may also be distributed in a network-connected computer system to store and execute computer-readable code in a distributed manner. In addition, functional programs, codes, and code segments for implementing the present embodiments may be easily understood by those skilled in the art to which the present embodiment belongs.

Although this application discussed above has been described with reference to the embodiments shown in the drawings, it is merely illustrative and will be understood by those skilled in the art that various modifications and variations of the embodiments are possible therefrom. However, such modifications should be considered to be within the technical protection scope of the present application. Therefore, the true technical protection scope of the present application should be determined by the technical spirit of the appended claims.

### INDUSTRIAL AVAILABILITY

The present application is intended to provide a method of obtaining, via a sensor array, the pre-food swallowing voice or vibration and the post-food swallowing voice or vibration of the subject, respectively, and providing auxiliary information on dysphagia by comparing one or more characteristic values of each of said voice or vibration.

In another aspect, the present application is intended to provide auxiliary information on the location or amount of the residue, in addition to aspiration.

According to the method for providing auxiliary information on dysphagia using such voice analysis, it is possible to accurately and effectively calculate auxiliary information, particularly information on the location or amount of residue, for determining whether there is a dysphagia by comparing each characteristic value of voice or vibration before and after the subject's food swallowing.

1. A method for providing auxiliary information on dysphagia using voice analysis, the method comprising:
obtaining, via a sensor array, the pre-food swallowing voice or vibration and the post-food swallowing voice or vibration of the subject, respectively, and providing auxiliary information on dysphagia by comparing one or more characteristic values of each of the voice or vibration;
wherein the auxiliary information on dysphagia comprises characteristic information about the residue state concerning one or more of the following: whether food has been aspirated into the airway after food swallowing by the subject, the presence or absence residues, the location of residues, and the amount of residues.

2. The method of claim 1, wherein:
each of the sensors of the sensor array is attached to a residue site of interest of the subject,
the step comprises:
calculating one or more characteristic values from each of the pre-food swallowing voice or vibration and the post-food swallowing voice or vibration of the subject obtained by each of the sensors by sites on the subject; and
comparing one or more characteristic values of each of the pre-swallowing voice or vibration and the post-swallowing voice or vibration obtained at the same site, to calculate characteristic information on the residue state, comprising one or more of following: whether food has been aspirated into the airway after the subject's food swallowing, the presence or absence of residues, the site of residues, and the amount of residues.

3. The method of claim 1, wherein:
each of sensors of the sensor array comprises at least a microphone.

4. The method of claim 3, wherein:
each of the sensors of the sensor array further comprises a vibration sensor that senses vibration.

5. The method of claim 2, wherein:
the method comprises,
providing further information on one or more of the following: whether food has been aspirated into the airway and whether residues remain in the pharynx, based on the characteristic information about the residue state.

6. The method of claim 2, wherein:
the method further comprises,
determining, based on the characteristic information on the residue state, whether food has been aspirated into the airway of the subject after the subject's food swallowing; and
determining, in response to determining that food has been aspirated into the airway of the subject, the amount of the aspirated food based on the characteristic information on the residue state.

7. The method of claim 6, wherein:
determining whether food has been aspirated into the airway of the subject comprises,
determining that food has been aspirated into the airway if the difference between one or more characteristic values of each of the pre-food swallowing voice or vibration and the post-food swallowing voice or vibration of the subject obtained by a sensor attached to the subject's oral cavity or airway proximal site is greater than or equal to a pre-specified first reference value.

8. The method of claim 5, wherein:
the method further comprises,
determining, based on the characteristic information on the residue state, whether residues remain in the pharynx of the subject after the subject's food swallowing; and
determining, in response to determining that residues remain in the pharynx of the subject, the amount of residues remaining in the pharynx based on the characteristic information on the residue state.

9. The method of claim 8, wherein:
determining whether residues remain in the pharynx of the subject comprises,
determining that residues remain in the pharynx of the subject if the difference between one or more characteristic values of the pre-food swallowing voice or vibration and the post-food swallowing voice or vibration of the subject obtained by the sensor attached to the subject's oral cavity or pharynx proximal site is greater than or equal to a pre-specified second reference value.

10. The method of claim 1, wherein:
the voice or vibration is a voice or vibration when the subject utters a specific word for a period of time.

11. The method of claim 1, wherein:
the one or more characteristic values are calculated using a PRAAT.

12. The method of claim 1, wherein:
the one or more characteristic values comprise one or more selected from the group consisting of average fundamental frequency (F0) for all extracted pitch periods of voice or vibration, standard deviation of the fundamental frequency, relative average perturbation (RAP), jitter, shimmer percentage, amplitude perturbation quotient (APQ), noise-to-harmonic ratio (NHR), harmonics to noise ratio (HNR), voice turbulence index (VTI), and signal to noise ratio (SNR).

13. (canceled)

14. (canceled)

15. The method of claim 12, wherein:
if the relative average perturbation (RAP) of the post-food swallowing voice or vibration increases or decreases compared to the pre-food swallowing voice or vibration obtained by the sensor attached to the same site, the residue location is determined to be near the sensor attachment site, and
it is determined that the amount of residue is large in the region close to the site where the RAP variability is greatest among the sites to which the sensor is attached, by comparing the RAP variability of the pre-food swallowing voice or vibration and the post-food swallowing voice or vibration obtained by each of the sensors by sites on the subject.

16. The method of claim 12, wherein:
if at least one of jitter, shimmer percentage, and voice turbulence index of the post-swallowing voice or vibration increases or decreases compared to the pre-food swallowing voice or vibration, the residue location is determined to be near the sensor attachment site, and
it is determined that the amount of residue is large in the region close to the site where the variability is greatest among the sites to which the sensor is attached, by comparing the variability of at least one of jitter, shimmer percentage, and voice turbulence index of the pre-food swallowing voice or vibration and the post-food swallowing voice or vibration obtained by each of the sensors by sites on the subject.

17. The method of claim 2, wherein:
the residue sites of interest are at least one or more of the subject's lips, the skin surface near the vallecular pouch, the skin surface near the vocal cord, the skin surface near the pyriformis sinus, and the skin surface near the pharyngeal wall, of the subject's neck.

18. The method of claim 1, wherein:
the method further comprises providing control information for an electrical stimulator to assist with dysphagia rehabilitation treatment of the subject, or providing guide information about food if the characteristic information on the residue state is determined to be within a pre-set range related to dysphagia, by obtaining the pre-food swallowing voice or vibration and the post-food swallowing voice or vibration of the subject via the sensor array, respectively, and comparing the one or more characteristic values of each of the voice or vibration.

19. The method of claim 18, wherein:
the method provides control information for the parameter values of a 4-channel electrostimulator, based on at least one of whether food has been aspirated into the airway, the amount of the aspirated food, whether residues remain in the pharynx, and the amount of residue remaining in the pharynx.

20. The method of claim 18, wherein:
the method provides guide information on the type, quality, and concentration of food, based on at least one of whether food has been aspirated into the airway, the amount of the aspirated food, whether residues remain in the pharynx, and the amount of residue remaining in the pharynx.

21. The method of claim 18, wherein:
the subject is a dysphagia patient, and
the auxiliary information on dysphagia comprises auxiliary information on the improvement of dysphagia.

22. The method of claim 1, wherein:
the method further comprises,
obtaining, via the sensor array, at least a first voice or vibration and a second voice or vibration, respectively, after subject's food swallowing, with the second voice or vibration obtained after a period of time has elapsed from the first voice or vibration; and
comparing one or more characteristic values of each of the pre-food swallowing voice or vibration, the first post-swallowing voice or vibration, and the second post-swallowing voice or vibration of the subject obtained at the same site to provide characteristic information on the residue state.

23. (canceled)
